(19) United States
(12) Patent Application Publication
(10) Pub. No.: US 2009/0060841 A1
(43) Pub. Date: Mar. 5, 2009

(54) APPARATUS AND METHOD FOR COMBINED USE OF VARIABLE FLIP ANGLES AND CENTRIC PHASE ENCODING IN HYPERPOLARIZED 13C IMAGING

(76) Inventors: Yi-Fen Yen, Menlo Park, CA (US); Ralph E. Hurd, Milpitas, CA (US); Susan J. Kohler, Niskayuna, NY (US)

Correspondence Address: ZIOLKOWSKI PATENT SOLUTIONS GROUP, SC (GEMS), 136 S WISCONSIN ST, PORT WASHINGTON, WI, 53074, US

(21) Appl. No.: 12/045,177

(22) Filed: Mar. 10, 2008

Related U.S. Application Data
(60) Provisional application No. 60/968,214, filed on Aug. 27, 2007.

(51) Int. Cl. A61B 5/055

(52) U.S. Cl. 424/9.3; 600/414

(57) ABSTRACT

A system and method for MR imaging includes a magnetic resonance imaging (MRI) system having a plurality of gradient coils positioned about a bore of a magnet, and an RF transceiver system and an RF switch controlled by a pulse module to transmit RF signals to an RF coil assembly to acquire MR images. The apparatus further includes a controller programmed to determine a variable flip angle (VFA) sequence to excite a hyperpolarized material in a subject and to determine a delay period during which application of the VFA sequence is delayed after injection of a hyperpolarized contrast agent. The delay period is based on dynamic data of the hyperpolarized material acquired from the subject. The controller is also programmed to cause application of the VFA sequence to excite the hyperpolarized material in the subject and to acquire MR data from the hyperpolarized material using an isotropic centric phase encoding (iCPE) technique.

### CROSS REFERENCE TO RELATED APPLICATION

The present application claims the benefit of U.S. Provisional Application Ser. No. 60/968,214, filed Aug. 27, 2007.

### BACKGROUND OF THE INVENTION

The invention relates generally to a system and method of utilizing a hyperpolarized signal in a gradient echo sequence for magnetic resonance (MR) imaging and in particular to using variable flip angles and phase encoding in centric order for hyperpolarized metabolic imaging.

When a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, or “longitudinal magnetization,” M_{Z}, may be rotated, or “tipped,” into the x-y plane to produce a net transverse magnetic moment Mₜ. A signal is emitted by the excited spins after the excitation signal B₁ is terminated and this signal may be received and processed to form an image.

When utilizing these signals to produce images, magnetic field gradients (Gₓ, G_{y}, and G_{z}) are employed. Typically, the region to be imaged is scanned by a sequence of measurement cycles in which these gradients vary according to the particular localization method being used. The resulting set of received NMR signals are digitized and processed to reconstruct the image using one of many well-known reconstruction techniques.

Magnetic resonance imaging of hyperpolarized ¹³C-labeled contrast agent allows imaging of both the contrast agent and the metabolized hyperpolarized products that derive from the contrast agent. In such an imaging session, the ¹³C-labeled contrast agent follows a metabolic pathway, and the intensity and spatial distribution of the labeled agent as well as its metabolic products may be imaged. The magnetization of a hyperpolarized agent is unrecoverable. Thus, once it is dissolved, its magnetization decays through T1 relaxation. Following an injection, the labeled agent (such as ¹³C-pyruvate) undergoes metabolic exchange into other metabolites, such as lactate, alanine, and bicarbonate. These metabolic products also carry a hyperpolarized ¹³C label and have different T1 relaxation rates. If the time window of image data acquisition is too early, the receiver may saturate due to the large pyruvate signal immediately following the injection. If the acquisition window is too late, the majority of the metabolic product signals may be missed. In addition, when applying multiple phase encodings, the k-space signal is modulated by the dynamic curve, and different curve shapes may result in different image artifacts.

It would therefore be desirable to have a system and method capable of utilizing a hyperpolarized signal in a gradient echo sequence.

### BRIEF DESCRIPTION OF THE INVENTION

In accordance with one aspect of the invention, a magnetic resonance imaging (MRI) apparatus includes a plurality of gradient coils positioned about a bore of a magnet, and an RF transceiver system and an RF switch controlled by a pulse module to transmit RF signals to an RF coil assembly to acquire magnetic resonance (MR) images. The apparatus further includes a controller programmed to determine a variable flip angle sequence to excite a hyperpolarized material in a subject, determine a delay period during which application of the variable flip angle sequence is delayed after injection of a hyperpolarized contrast agent, the delay period based on dynamic data of the hyperpolarized material acquired from the subject, cause application of the variable flip angle sequence to excite the hyperpolarized material in the subject, and acquire MR data from the hyperpolarized material using an isotropic centric phase encoding technique.

According to another aspect of the invention a method of MR imaging includes injecting a hyperpolarized contrast agent into a subject, delaying after the injection for a period that is based on dynamic data of metabolism of the contrast agent within the subject, applying a variable flip angle sequence to excite the hyperpolarized materials in the subject, and acquiring MR data from the hyperpolarized materials using an isotropic centric phase encoding technique.

According to yet another aspect of the invention, a computer program includes instructions which when executed by a computer cause the computer to determine a succession of variable flip angles to be applied in the MRI sequence. The sequence is applied to excite a hyperpolarized substance in a subject, at a time delay following an injection of hyperpolarized contrast agent, and acquire MR data from the hyperpolarized substance using an isotropic centric phase encoding technique.

Various other features and advantages will be made apparent from the following detailed description and the drawings.

### BRIEF DESCRIPTION OF THE DRAWINGS

The drawings illustrate embodiments presently contemplated for carrying out the invention.

In the drawings:

FIG. 1 is a schematic block diagram of an exemplary MR imaging system for use with embodiments of the invention.

FIG. 2 is a technique for acquiring MR data using a hyperpolarized agent according to an embodiment of the invention.

FIG. 3 is a graph showing an example of dynamic curves acquired in vivo following an injection of hyperpolarized ¹³C-pyruvate.

FIGS. 4-6 illustrate simulated trends of pyruvate, lactate, and alanine from data within imaging windows 201, 203, and 205 of FIG. 3, respectively, that were simulated based on an embodiment of the invention.

### DETAILED DESCRIPTION OF THE PREFERRED EMBODIMENT

A system is shown to utilize a hyperpolarized signal in a gradient echo sequence for MR imaging. Variable flip angles and phase encoding in centric order are combined to improve the signal to noise ratio and image quality of hyperpolarized metabolic imaging.

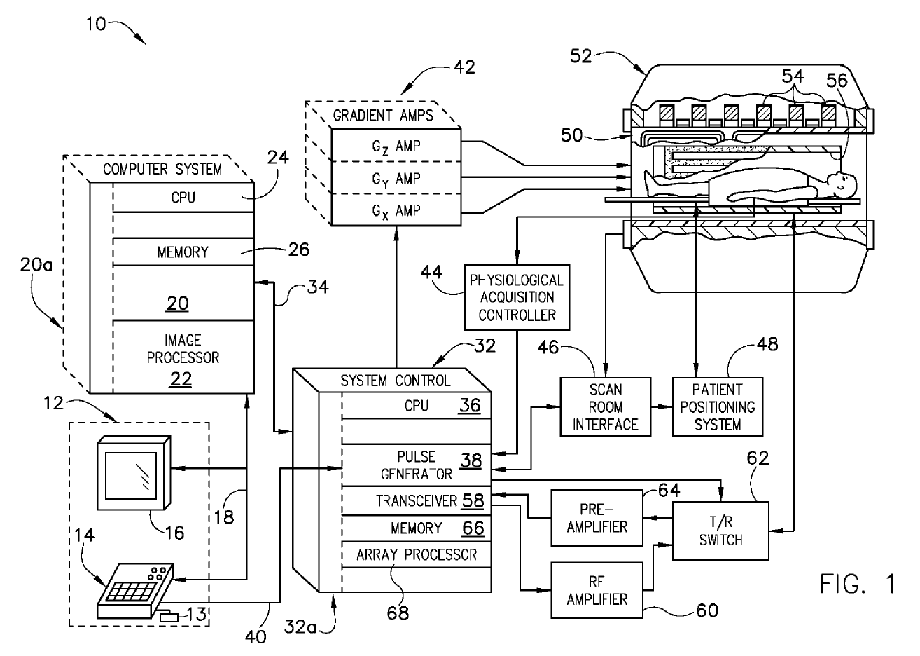

Referring to FIG. 1, the major components of an exemplary magnetic resonance imaging (MRI) system 10 incorporating embodiments of the invention are shown. The operation of the system is controlled from an operator console 12 which includes a keyboard or other input device 13, a control panel 14, and a display screen 16. The console 12 communicates through a link 18 with a separate computer system 20 that enables an operator to control the production and display of images on the display screen 16. The computer system 20 includes a number of modules which communicate with each other through a backplane 20a. These include an image processor module 22, a CPU module 24 and a memory module 26 that may include a frame buffer for storing image data arrays. The computer system 20 is linked to archival media devices, permanent or back up memory or a network for storage of image data and programs, and communicates with a separate system control 32 through a high speed serial link 34. The input device 13 can include a mouse, joystick, keyboard, track ball, touch activated screen, light wand, voice control, or any similar or equivalent input device, and may be used for interactive geometry prescription.

The system control 32 includes a set of modules connected together by a backplane 32a. These include a CPU module 36 and a pulse generator module 38 which connects to the operator console 12 through a serial link 40. It is through link 40 that the system control 32 receives commands from the operator to indicate the scan sequence that is to be performed. The pulse generator module 38 operates the system components to carry out the desired scan sequence and produces data which indicates the timing, strength and shape of the RF pulses produced, and the timing and length of the data acquisition window. The pulse generator module 38 connects to a set of gradient amplifiers 42, to indicate the timing and shape of the gradient pulses that are produced during the scan. The pulse generator module 38 can also receive patient data from a physiological acquisition controller 44 that receives signals from a number of different sensors connected to the patient, such as ECG signals from electrodes attached to the patient. And finally, the pulse generator module 38 connects to a scan room interface circuit 46 which receives signals from various sensors associated with the condition of the patient and the magnet system. It is also through the scan room interface circuit 46 that a patient positioning system 48 receives commands to move the patient to the desired position for the scan.

The gradient waveforms produced by the pulse generator module 38 are applied to the gradient amplifier system 42 having Gx, Gy, and Gz amplifiers. Each gradient amplifier excites a corresponding physical gradient coil in a gradient coil assembly generally designated 50 to produce the magnetic field gradients used for spatially encoding acquired signals. The gradient coil assembly 50 forms part of a magnet assembly 52 which includes a polarizing magnet 54 and a whole-body RF coil 56. A transceiver module 58 in the system control 32 produces pulses which are amplified by an RF amplifier 60 and coupled to the RF coil 56 by a transmit/receive switch 62. The resulting signals emitted by the excited nuclei in the patient may be sensed by the same RF coil 56 and coupled through the transmit/receive switch 62 to a preamplifier 64. The amplified MR signals are demodulated, filtered, and digitized in the receiver section of the transceiver 58. The transmit/receive switch 62 is controlled by a signal from the pulse generator module 38 to electrically connect the RF amplifier 60 to the coil 56 during the transmit mode and to connect the preamplifier 64 to the coil 56 during the receive mode. The transmit/receive switch 62 can also enable a separate RF coil (for example, a surface coil) to be used in either the transmit or receive mode.

The MR signals picked up by the RF coil 56 are digitized by the transceiver module 58 and transferred to a memory module 66 in the system control 32. A scan is complete when an array of raw k-space data has been acquired in the memory module 66. This raw k-space data is rearranged into separate k-space data arrays for each image to be reconstructed, and each of these is input to an array processor 68 which operates to Fourier transform the data into an array of image data. This image data is conveyed through the serial link 34 to the computer system 20 where it is stored in memory. In response to commands received from the operator console 12, this image data may be archived in long term storage or it may be further processed by the image processor 22 and conveyed to the operator console 12 and presented on the display 16.

MR spectroscopic imaging using hyperpolarized ¹³C-pyruvate is a technique for imaging that uses mapped metabolic activity in vivo and has the potential for applications in cardiac function, brain perfusion, and prostate cancer detection. The largely enhanced ¹³C MR signal from hyperpolarized ¹³C-pyruvate allows for ¹³C imaging of a 3D volume with good spatial resolution. The MR spectroscopic imaging technique images not only the injected contrast agent but also its metabolic products, such as lactate, alanine, and bicarbonate. Therefore, it allows observation of the metabolic function of organs and can provide high specificity in some diagnoses.

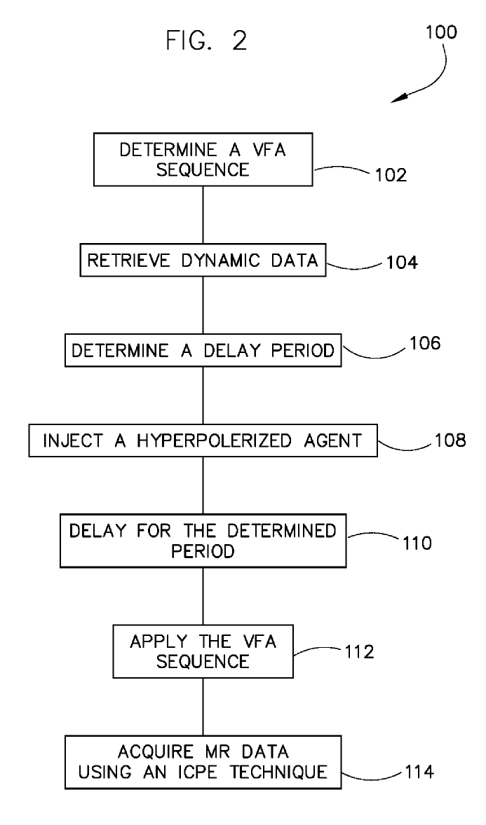

FIG. 2 shows an MR spectroscopic imaging technique for acquiring MR data using a hyperpolarized agent according to an embodiment of the invention. Technique 100 begins at block 102 where a variable flip angle (VFA) sequence is determined to excite a hyperpolarized material in the subject. A VFA sequence uses a series of progressively increasing flip angles up to 90° for imaging with multiple excitations. The VFA sequence is suited to hyperpolarized imaging because the pre-polarized magnetization undergoes T1 relaxation and is unrecoverable. Imaging using a VFA sequence also typically utilizes substantially all longitudinal magnetization and optimizes the signal-to-noise ratio.

When T1 is known, the design of a VFA sequence typically incorporates the T1 value and, hence, tends to yield constant transverse magnetization at each excitation and eliminate image artifacts due to T1 modulation among phase encodings. However, because the in vivo T1 of ¹³C metabolites is unknown and metabolites undergo different dynamics the VFA schedule is designed by mathematically assuming that T1 is infinite and that either there is no metabolic exchange or the metabolic exchange has reached a steady state according to an embodiment of the invention. Then,

θₙ=tan⁻¹(sin(θₙ₊₁)), (Eqn. 1),

where θₙ is the flip angle of the nth excitation, and the flip angle of the last excitation is 90°. In order to completely utilize the hyperpolarized signal in gradient echo sequences, the VFA sequence is designed to divide the magnetization at each excitation. As will be described, the magnetization products at each excitation are similar because the imaging window is chosen to be on the plateau of their dynamic curves.

At block 104 of FIG. 2, dynamic data for a contrast agent, such as ¹³C-pyruvate, and its metabolic products are obtained or retrieved from files having dynamic data taken during a previous acquisition from the same or a different subject. In one embodiment, dynamic data is acquired from one or more different subjects having a similar tissue type and disease condition.

Optimizing signal and image quality in ¹³C metabolic imaging includes accounting for different signal time curves or dynamic curves for the different ¹³C metabolites. Typically, the ¹³C-pyruvate signal increases rapidly during an injection, peaks shortly after the end of injection, and then slowly decays monotonically due to T1 relaxation and exchange into metabolic products. Recirculation may be visible in the pyruvate dynamic curves in some cases where a large dose is injected. The metabolic dynamic curves typically increase slowly, reach a quasi-steady state for about 12-15 seconds, and then decay. The shape of the metabolite or product curves is the result of competing processes between T1 relaxation and metabolic exchange with pyruvate.

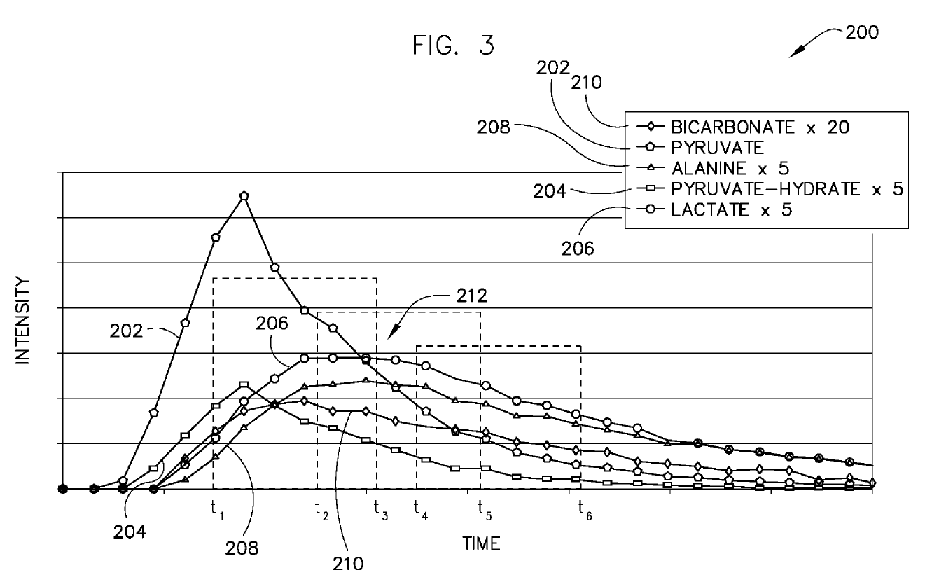

FIG. 3 shows an example of dynamic curves acquired in one slice following an injection of hyperpolarized ¹³C-pyruvate and is representative of data that may be used according to embodiments of the invention. Graph 200 includes pyruvate as a labeled agent at 202, pyruvate-hydrate at 204, and metabolite signals 206-210 that include, respectively, lactate 206, alanine 208, and bicarbonate 210.

In order to acquire MR imaging data, imaging is typically started before or while the metabolite, such as lactate, reaches its plateau. Depending on the total scan time required by the imaging sequence, the VFA sequence results in a nearly constant lactate signal in the beginning of imaging and in a slightly decreased signal toward the end of imaging. During this imaging period, the pyruvate signal is typically completely utilized by the VFA sequence, and the signal decreases monotonically from excitation to excitation.

Thus, as illustrated in FIG. 3, when applying a VFA sequence as described above, imaging data may be acquired during the plateau 212 of, for instance, the lactate dynamic curve 206 when a fairly constant lactate transverse magnetization from excitation to excitation occurs. The temporal resolution of the curves 402-410 illustrated is approximately 3 seconds, and the injection duration is approximately 12 seconds. The hyperpolarized pyruvate signal 202 peaks at approximately 15-18 seconds after the start of injection and then promptly decays. Hyperpolarized metabolite signals of lactate 206, alanine 208, and bicarbonate 210 begin to appear approximately 6-8 seconds after the start of injection, plateau (or acquire peak concentration or production) for approximately 12 seconds, and then decay slowly.

Therefore, based on the data illustrated in FIG. 3, imaging data may be obtained during a period when the hyperpolarized pyruvate signal is finite but decaying and when the hyperpolarized metabolite signal is plateauing as well. Thus, referring back to FIG. 2, at block 104, dynamic data is acquired, which typically includes, according to an embodiment of the invention, pyruvate data and at least one metabolite such as lactate, alanine, and bicarbonate.

Referring again to FIG. 2, a delay period is determined based on the acquired or retrieved dynamic data for the hyperpolarized material at block 106. Because of the dynamic behavior of both the contrast agent and metabolic products thereof, the delay period is determined such that a ¹³C-pyruvate signal is present after an injection, and metabolic product signals are, likewise, present. At block 108, the subject is injected with a hyperpolarized agent, such as ¹³C-pyruvate. At block 110, the delay period determined at block 106 is implemented, and at block 112, the VFA sequence is applied.

After the VFA sequence is applied at block 112, MR data is accordingly acquired at block 114. In order to further optimize the pyruvate signal-to-noise ratio (SNR) by taking advantage of the relatively high pyruvate signal at the beginning of acquisition, a phase encoding (PE) sequence may be performed in concentric order according to an embodiment of the invention. The order of encoding is prioritized according to the distance (1/cm) between each k-space sample point to the origin. When more than one dimension of k-space is sampled, sorting is applied to sample points in all dimensions strictly according to their distances to the origin. This sorting method allows a more isotropic sampling pattern in k-space even when the field of view (FOV) of each imaging dimension is different. The isotropic centric phase encoding (iCPE) sequence eliminates image blurring caused by signal modulation of underlying dynamics.

As stated above, MR data is acquired from the subject at block 114. According to an embodiment of the invention, MR data is acquired using an iCPE sequence. The iCPE sequence is used in order to sample the relatively high signal at the origin of k-space. The gradient echo sequence data acquired may be 3D echo-planar spectroscopic imaging (3DEPSI) or a fast chemical shift imaging (fastCSI) sequence; however, one skilled in the art will recognize that other sequences may be applied as well.

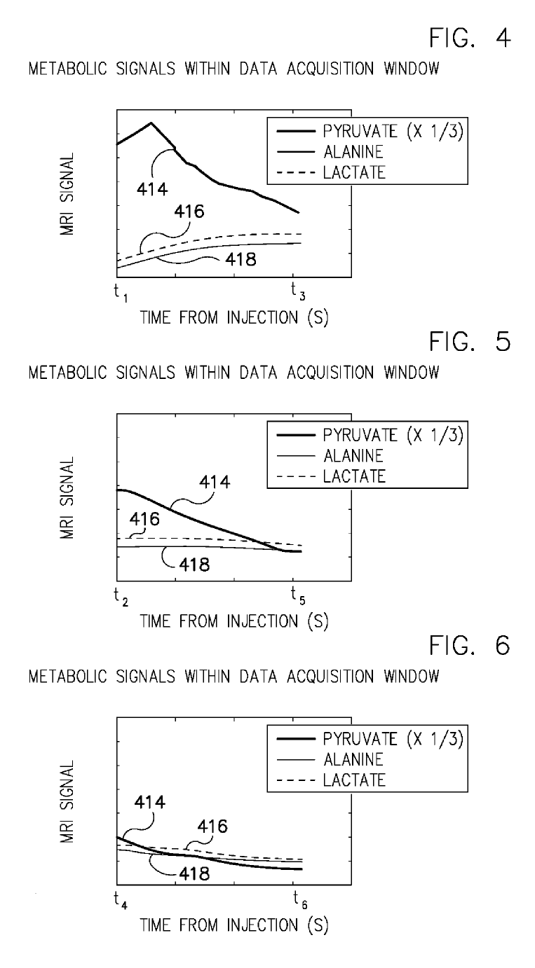

FIGS. 4-6 illustrate simulated trends of pyruvate, lactate, and alanine from data within imaging windows 201, 203, and 205 of FIG. 3, respectively, that were simulated based on an embodiment of the invention. FIG. 4 shows that at time, t₁, the receiver may saturate due to the high pyruvate trend 414. Curves for lactate 416 and alanine 418 in FIG. 4 show a trend increase in their MRI signal from time, t₁, to time, t₃. FIGS. 5 and 6 illustrate that the trends for lactate 416 and alanine 418 are at quasi-steady state, while the trend for pyruvate 414 decreases relatively rapidly and monotonically. Accordingly, a time delay of t₂ or t₄, for example, allows the metabolite signals 416, 418 to be substantially fully utilized.

An embodiment of the invention provides that the ¹³C MR signal from hyperpolarized ¹³C-pyruvate allows ¹³C imaging of 3D volume with good spatial resolution. Also, an embodiment of the invention images not only the injected contrast agent but also its metabolic products, thereby allowing observation of the metabolic function of organs.

Therefore, according to an embodiment of the invention a magnetic resonance imaging (MRI) apparatus includes a plurality of gradient coils positioned about a bore of a magnet, and an RF transceiver system and an RF switch controlled by a pulse module to transmit RF signals to an RF coil assembly to acquire magnetic resonance (MR) images. The apparatus further includes a controller programmed to determine a variable flip angle sequence to excite a hyperpolarized material in a subject, determine a delay period during which application of the variable flip angle sequence is delayed after injection of a hyperpolarized contrast agent, the delay period based on dynamic data of the hyperpolarized material acquired from the subject, cause application of the variable flip angle sequence to excite the hyperpolarized material in the subject, and acquire MR data from the hyperpolarized material using an isotropic centric phase encoding technique.

According to another embodiment of the invention a method of magnetic resonance (MR) imaging includes injecting a hyperpolarized contrast agent into a subject, delaying after the injection for a period that is based on dynamic data of metabolism of the contrast agent within the subject, applying a variable flip angle sequence to excite a hyperpolarized material in the subject, and acquiring MR data from the hyperpolarized material using an isotropic centric phase encoding technique.

According to yet another embodiment of the invention, a computer program includes instructions which when executed by a computer cause the computer to determine a succession of variable flip angles to excite a hyperpolarized substance in a subject, implement a delay period during a magnetic resonance (MR) imaging session, the delay period based on dynamic data of the hyperpolarized substance acquired from the subject, cause application of the succession of variable flip angles after the delay period to excite the hyperpolarized substance in the subject, and acquire MR data from the hyperpolarized substance using an isotropic centric phase encoding technique.

A technical contribution for the disclosed method and apparatus is that it provides for a computer implemented use of a hyperpolarized signal in a gradient echo sequence for MR imaging. Variable flip angles and phase encoding in centric order are combined to improve the SNR and image quality of hyperpolarized metabolic imaging.

The invention has been described in terms of the preferred embodiment, and it is recognized that equivalents, alternatives, and modifications, aside from those expressly stated, are possible and within the scope of the appending claims.

1-21. (canceled)

22. An MRI apparatus comprising:
a magnetic resonance imaging (MRI) system having a plurality of gradient coils positioned about a bore of a magnet, and an RF transceiver system and an RF switch controlled by a pulse module to transmit RF signals to an RF coil assembly to acquire magnetic resonance (MR) images; and
a controller programmed to:
determine a variable flip angle sequence to excite a hyperpolarized material in a subject;
determine a delay period during which application of the variable flip angle sequence is delayed after injection of a hyperpolarized contrast agent, the delay period based on dynamic data of the hyperpolarized material;
cause application of the variable flip angle sequence to excite the hyperpolarized material in the subject; and
acquire MR data from the hyperpolarized material using an isotropic centric phase encoding technique.

23. The apparatus of claim 22 wherein the hyperpolarized material comprises the hyperpolarized contrast agent.

24. The apparatus of claim 23 wherein the MR data is acquired during a decay period of the hyperpolarized contrast agent.

25. The apparatus of claim 22 wherein the variable flip angle sequence is determined using a mathematical assumption that a T1 parameter of the hyperpolarized material is infinite.

26. The apparatus of claim 22 wherein the hyperpolarized material is a metabolite of the hyperpolarized contrast agent.

27. The apparatus of claim 26 wherein the MR data is acquired during a time period when a peak concentration of the metabolite is present in the subject.

28. The apparatus of claim 26 wherein the metabolite is produced in vivo from the hyperpolarized contrast agent.

29. The apparatus of claim 26 wherein the metabolite includes at least one of lactate, alanine, and bicarbonate.

30. The apparatus of claim 22 wherein the hyperpolarized contrast agent is ¹³C pyruvate.

31. The apparatus of claim 22 wherein the computer is further caused to apply a gradient echo sequence that includes one of a 3D echo-planar spectroscopic imaging (3DEPSI) sequence and a fast Chemical Shift Imaging (fastCSI) sequence.

32. A method of magnetic resonance (MR) imaging comprising:
injecting a hyperpolarized contrast agent into a subject;
delaying after the injection for a period that is based on dynamic data of metabolism of the contrast agent;
applying a variable flip angle sequence to excite a hyperpolarized material in the subject; and
acquiring MR data from the hyperpolarized material using an isotropic centric phase encoding technique.

33. The method of claim 32 wherein the hyperpolarized contrast agent is ¹³C pyruvate.

34. The method of claim 32 wherein the hyperpolarized material is the hyperpolarized contrast agent.

35. The method of claim 32 wherein the hyperpolarized material is a metabolite of the hyperpolarized contrast agent.

36. The method of claim 35 wherein the metabolite is one of lactate, alanine, and bicarbonate.

37. The method of claim 35 wherein the step of acquiring further comprises acquiring the imaging data during a peak production of the metabolite.

38. A computer readable storage medium having stored thereon a computer program comprising instructions which when executed by a computer cause the computer to:
determine a succession of variable flip angles to excite a hyperpolarized substance in a subject;
implement a delay period during a magnetic resonance (MR) imaging session, the delay period based on dynamic data of the hyperpolarized substance;
cause application of the succession of variable flip angles after the delay period to excite the hyperpolarized substance in the subject; and
acquire MR data from the hyperpolarized substance using an isotropic centric phase encoding technique.

39. The computer readable storage medium of claim 38 wherein the hyperpolarized substance is an injected hyperpolarized contrast agent.

40. The computer readable storage medium of claim 39 wherein the injected hyperpolarized contrast agent is ¹³C pyruvate.

41. The computer readable storage medium of claim 38 wherein the hyperpolarized substance is a metabolic product of an injected hyperpolarized contrast agent.

42. The computer readable storage medium of claim 41 wherein the metabolic product signal derives from one of lactate, alanine, and bicarbonate.
